(19) United States
(12) Patent Application Publication
(10) Pub. No.: US 2013/0102863 A1
(43) Pub. Date: Apr. 25, 2013

(54) Non-Invasive Trans-Reflective Monitoring Apparatus

(71) Applicant: Gerard Aknine, Gannes (FR)

(72) Inventor: Gerard Aknine, Gannes (FR)

(21) Appl. No.: 13/481,689

(22) Filed: May 25, 2012

Related U.S. Application Data
(60) Provisional application No. 61/489,985, filed on May 25, 2011.

(51) Int. Cl. A61B 5/1455

(52) U.S. Cl. CPC A61B 5/14552
USPC 600/323

(57) ABSTRACT

Described herein are apparatus, devices and methods of use to monitor a patient's condition. The apparatus includes a housing having an internal volume divided into a first and second cavity by a central opaque partition; a light source positioned within the first cavity and coupled to a first side of the partition; a detector positioned within the second cavity and coupled to a second side of the partition; and a lens material filling the internal volume of the housing and enclosing the first and second cavities. The light source includes a first light emitting diode configured to emit a first wavelength of light and a second light emitting diode configured to emit a second wavelength that is different from the first wavelength of light. The detector is configured to measure at least one of an absorbance, a scattering or a frequency of the first and second wavelengths of light.

### CROSS REFERENCE TO RELATED APPLICATIONS

This application claims the benefit of priority under 35 U.S.C. §119 to U.S. Provisional Patent Application Ser. No. 61/489,985, filed on May 25, 2011, entitled, “NON-INVASIVE TRANS-REFLECTIVE MONITORING APPARATUS”, the entire disclosures of which is incorporated by reference herein.

### FIELD

This disclosure relates generally to instruments and methods for non-invasively monitoring the status of a patient.

### BACKGROUND

Medical personnel monitor blood flow characteristics by pulse oximetry, which can measure arterial hemoglobin oxygen saturation by use of a non-invasive probe.

### SUMMARY

The subject matter disclosed herein provides methods and devices, for the non-invasive monitoring of blood oxygenation and other vital signs.

In one aspect there is provided an apparatus including a housing having an internal volume divided into a first and second cavity by a central opaque partition; a light source positioned within the first cavity of the housing and coupled to a first side of the partition; a detector positioned within the second cavity of the housing and coupled to a second side of the partition; and a lens material filling the internal volume of the housing and enclosing the first and second cavities. The light source includes a first light emitting diode configured to emit a first wavelength of light and a second light emitting diode configured to emit a second wavelength that is different from the first wavelength of light. The detector is configured to measure at least one of an absorbance, a scattering or a frequency of the first and second wavelengths of light. The first wavelength of light can be between about 660 nanometers to about 735 nanometers and emitted at a first frequency. The second wavelength of light can be between about 910 nanometers to about 990 nanometers and emitted at a second frequency that is different from the first frequency. The lens material can include an elastomeric lens material or a dielectric lens material.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWING

These and other aspects will now be described in detail with reference to the following drawings.

FIG. 1 depicts a perspective schematic view of a monitoring device;

FIG. 2 depicts a cross-sectional schematic view of the monitoring device of FIG. 1;

FIG. 3 depicts a bottom schematic view of the monitoring device of FIG. 1; and

FIG. 4 depicts an implementation of a monitoring device.

### DETAILED DESCRIPTION

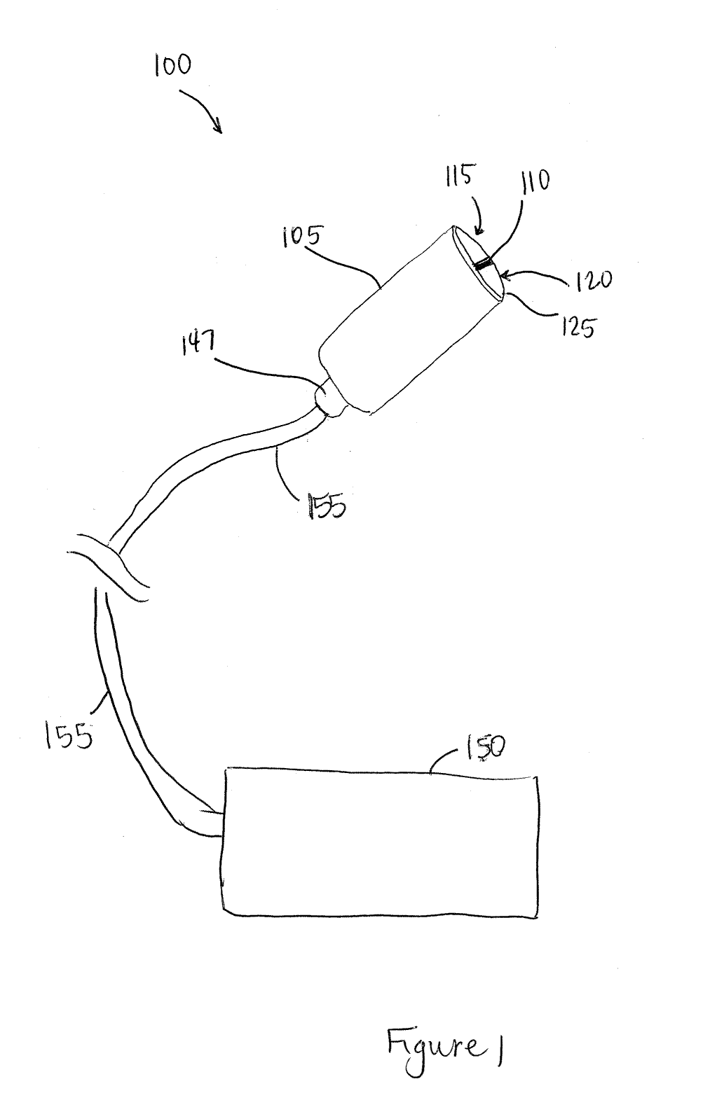

FIG. 1 depicts a perspective view of a monitoring device. The monitoring device 100 can include a housing 105 having an internal volume divided by an opaque partition 110 into a light source cavity 115 and a detector cavity 120. The internal volume of the housing 105 can be filed by a lens material 125. As will be described in more detail below, the monitoring device 100 also can include one or more sensors for monitoring a condition of a patient, including arterial hemoglobin oxygen saturation.

The monitoring device 100 can be used at any location in or on the body where monitoring of a condition of a patient would be desirable. In one implementation, the monitoring device 100 can be introduced through the vaginal canal and applied through a partially dilated cervix to a presenting part of the fetus while still in utero. The monitoring device 100 can monitor the condition of a fetus during the peripartum process, such as fetal heart rate, arterial hemoglobin oxygen saturation, electrical activity of the heart, or a combination thereof, by applying or pressing to the scalp of the fetus or any fetal presenting part. The devices described herein can be used to avoid low oxygen saturation of a fetus during labor and therein prevent the sequelae of fetal hypoxia and acidemia and issues with fetal encephalopathy.

It should be appreciated that use of the device is not limited to a particular anatomical region and that the device 100 can be used in a variety of locations on a patient's body as well as internal to a patient's body.

Conventional pulse oximetry uses a sensor placed on a thin part of a patient's body, such as a finger, toe, ear lobe, nose or scalp fold. The sensors can include a pair of small LEDs and a photodetector. The LEDs sequentially pass light in red wavelengths and infrared wavelengths to estimate the oxygen saturation and pulse rate from changes in absorption of the light detected throughout the blood pulse cycles. The technology is based on the differential absorbance of different wavelengths of light by different species of hemoglobin. Conventional pulse oximeters can be configured for transmittance or reflectance. Transmittance, or trans-illumination oximetry, involves the process whereby a sensor measures light extinction as light passes through a portion of blood-perfused tissue. Light is transmitted from one side of a portion of blood-perfused tissue, and is recorded by a photodetector situated across the portion of tissue. Reflectance oximetry has both the light source and the photodetector on one side of the tissue and measures reflectance back from the tissue. Reflectance oximetry generally has the light source and the photodetector in the same plane and in direct contact with the skin.

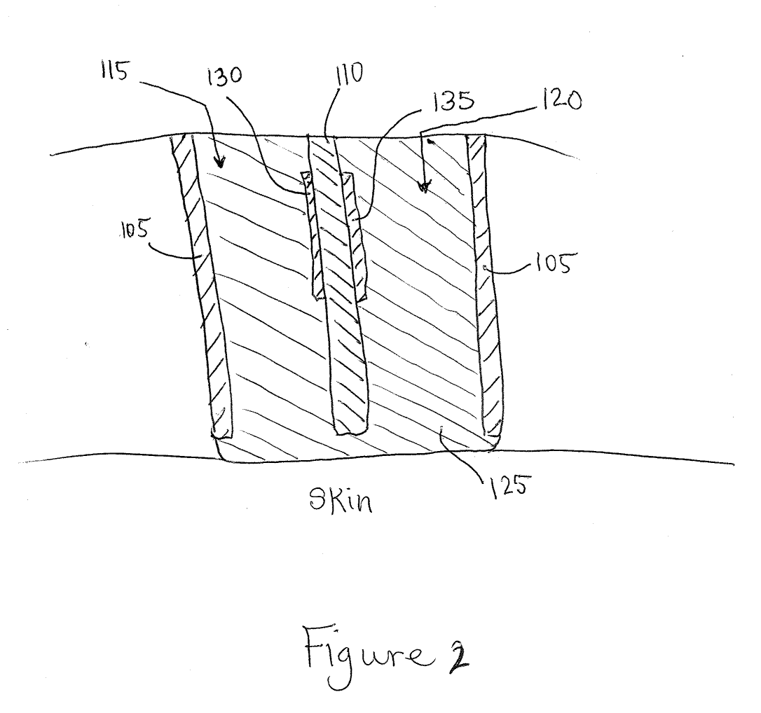

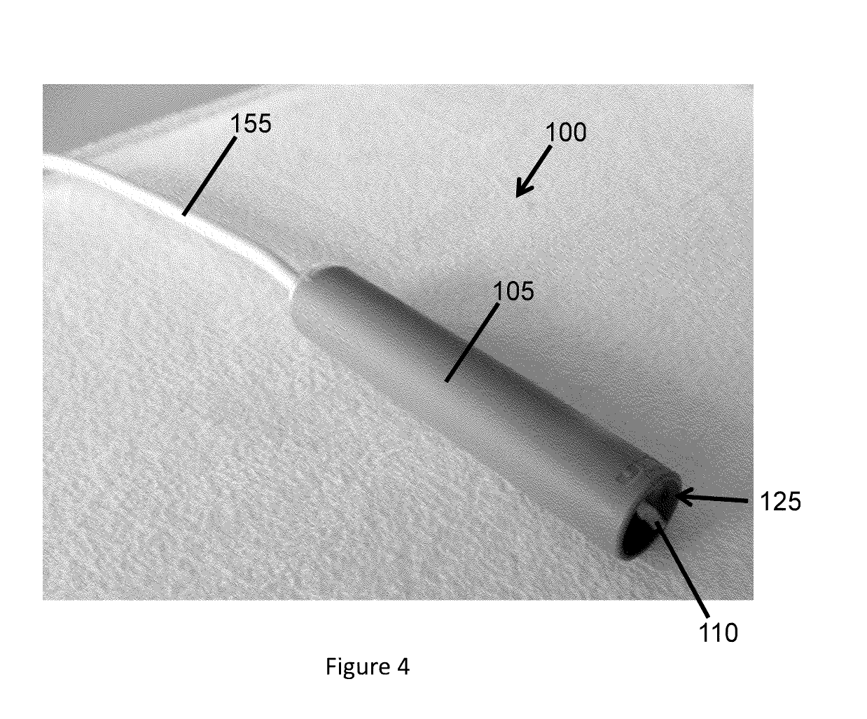

Described herein is a “trans-reflective” pulse oximetry device. The devices described herein can transmit light through a cavity and direct it towards the skin. The devices described herein also can detect reflected light that travels from the skin through a separate cavity. As best shown in FIG. 2 and also FIG. 4, the internal volume of the housing 105 is divided by a partition 110 into a light source cavity 115 and a detector cavity 120. The light source cavity 115 can include at least one light source 130 and the detector cavity 120 can include at least one detector 135.

In some implementations, the partition 110 is a printed circuit board (PCB). The PCB partition 110 can be printed on two sides such that one side includes the light source 130 and the opposite side includes the detector 135. As such, the PCB partition 110 can be configured to both carry and power the light source 130 and the detector 135. In this implementation, the light source 130 and detector 135 are positioned in a back-to-back configuration on the PCB partition 110 as shown in FIG. 2. A cable 155 can insert from a proximal end 147 of the housing 105, split to either side of the PCB partition 110 and directly connect to the light source 130 on a first side of the PCB partition 110 or the detector 135 on the second, opposite side of the PCB partition 110. The components can be external to the PCB partition 110. The PCT partition 110 can be covered by a light absorbing material.

It should be appreciated that other configurations are possible. For example, the light source 130 can be positioned on an internal wall of the housing 105 opposite the partition 110 within the light source cavity 115. Similarly, the detector 135 can be positioned on an internal wall of the housing 105 opposite the partition 110 within the detector cavity 120. It should be appreciated that the light source 130 and the detector 135 can be positioned within their respective cavities 115, 120 in a variety of configurations.

The partition 110 and the housing 105 can each be formed of an opaque material. The partition 110 can be an opaque material that can prevent detection of light emitted directly from the light source 130 that has not first been transmitted into and reflected from the vascular bed. The partition 110 can be a light absorbing color such as black whereas the housing 105 can be a light reflecting color such as white. The partition 110 and the housing 105 can be the same or a different material. The partition 110 and/or housing 105 can be formed of a polymer, fiberglass, ceramic, or other material or combination of materials. As described above, the partition 110 can be a printed circuit board.

As mentioned above, the light source cavity 115 and the detector cavity 120 can be filled with a lens material 125. The lens material 125 can guide light emitted from the light source 130 and transmit the light through the light cavity 115 towards the patient skin surface. The lens material 125 can concentrate the light into a narrow light beam that is directed towards the patient in a unidirectional manner. The lens material 125 also can guide light reflected from the patient through the detection cavity 125 to the detector 135. The lens material 125 can be an elastomeric material. The lens material 125 can be a dielectric material. The lens material 125 can be silicone, such as a transparent, translucent and/or colored silicone. In some implementations, the lens material 125 can be red-colored silicone such that the lens material 125 allows for greater transmission of red and infrared wavelengths through the lens material 125 towards the patient (or towards the detector 135) and prevents passage of light at other wavelengths (e.g. ambient light) that can interfere with a reading. The device 100 can also include a filter for the light detector 135 such as a red filter to avoid interference and improve accuracy of readings at wavelengths being used.

In addition to a light guiding function, the lens material 125 can also seal the device 100 and the electronic components of the device 100. The sealed device 100 can function properly to obtain accurate readings even on wet surfaces as well as when completely immersed in a fluid, as will be discussed in more detail below.

It should be appreciated that the volume of the lens material 125 can vary depending on the dimensions of the housing 105, which can also vary. In some implementations, the internal cavity of the housing 105 can be between about 30 mm to about 90 mm or greater in length. In some implementations, the housing 105 can have an outer diameter of between about 10 mm to about 30 mm. In some implementations, the internal cavity of the housing 105 can have an inner diameter of between about 8 mm to about 28 mm. It should be appreciated that the dimensions provided herein are for example only and can be changed. In addition, the housing 105 is shown in the figures as being a generally cylindrical element, but it should be appreciated that the shape of the housing 105 can vary, including rectangular or other shapes. In some implementations, the housing 105 can have a length and an outer diameter that allows it to be comfortably and conveniently inserted through a vaginal canal such that the distal end region of the housing 105 can insert through at least a portion of a minimally-dilated cervix.

The lens material 125 can fill the light cavity 115 and the detector cavity 125. The lens material 125 can also extend slightly beyond the distal end of the housing 105 forming a “bubble” or projection of lens material 125 at a distal end of the device 100. The projection of lens material 125 can form a generally curved upper surface at the distal end of the housing 105 that provides a smooth, soft surface for applying to a patient's skin. The thickness of the projection of lens material 125 extending distal to the housing 105 can vary. In some implementations, the thickness can be 0.5 mm, 0.75 mm, 1.0 mm, 1.25 mm, 1.5 mm or greater thickness. The lens material 125 projecting from the distal end of the housing 105 can have a surface area that is large enough to obtain an accurate reading, but not so large so as to become cumbersome. The lens material 125 projecting from the distal end of the housing 105 can be compressed and/or conformed somewhat to the tissue surface against which the monitoring device 100 is applied. In some implementations, the lens material 125 can be about 3 Shore A. In some implementations, the lens material 125 can be urged against the skin and compressed until it conforms to the shape of the tissue. In this example, infiltration of ambient light is reduced or prevented because the lens material 125 compresses until the opaque housing 105 closely surrounds the portion of skin through which the reading is being taken.

The light source 130 can include one or more light emitting diodes (LED) configured to emit light at a selected wavelength. In one aspect, the wavelength of one LED is in the red wavelength and the wavelength of the other LED is in the near infrared wavelength. In one aspect, the wavelength of one LED is about 660 nm and the wavelength of the other LED is about 940 nm. In another aspect, the wavelength of one LED is between about 660 nm to about 735 nm. In this aspect, the wavelength of the other LED is between about 910 nm to about 990 nm. In another aspect, the wavelength of one LED is about 730 nm and the wavelength of a second LED is about 910 nm. It should be appreciated that the light source 130 can include additional LEDs that emit additional wavelengths of light, such as a third wavelength of light. The LEDs can transmit large intensities of light at these different wavelengths at different frequencies. The LEDs can transmit the different frequencies of light in a simultaneous manner. The LEDs can also transmit the different wavelengths of light in a pulsatile, alternating manner such that the light is sampled by the detector 135 to measure the absorbances of the wavelengths of light absorbed by the tissue through which it is transmitted and reflected back to the detector 135.

The detector 135 can include one or more silicon photodiodes that produce current linearly proportional to the intensity of light striking it. The detector 135 can detect the absorption and/or scattering of the light from the tissue as well as the frequency of the light emitted from the light source 130.

As mentioned above, the light source 130 can transmit the light of different wavelengths simultaneously and at different frequencies. The detector 135 can discriminate between the frequency of the light emitted (e.g. 1 MHz vs. 2 MHz). As an example, one of the LEDs can emit light in the red wavelength at a first frequency (e.g. 1 MHz) and a second LED can emit light in the near infrared at a second higher frequency (e.g. 2 MHz). The red LED can be identified by the detector 135 by its first frequency and the near infrared LED can be identified by the detector 135 by its second, higher frequency. Because the LEDs can be identified by their higher or lower frequencies by the detector, the signals can be sent simultaneously and interference eliminated.

Unlike conventional pulse oximeters, the devices described herein have very high isolation, very high gain and transmit light through the cavities in a highly directional manner. In addition, the lens material 125 can seal the device 100 and all the electronic components contained within the housing 105. As such, the devices described herein can be used when wet or on wet surfaces as well as be fully immersed in a fluid and still function properly to provide accurate, consistent readings of the oxygen saturation and heart beat. The device 100 can be water-proof and/or water-resistant. In addition to maintaining function when wet or submerged, the device 100 also remains safe for a patient.

Unlike conventional pulse oximeters, the devices described herein need not be in direct contact with the patient's skin in order to obtain an accurate, consistent reading also due to their being highly directional and having very high gain. The device 100 can be positioned 1 mm, 2 mm, 3 mm or more away from the skin surface and still obtain accurate oxygen saturation and heart beat readings. The light cavity 115 in coordination with the partition 110 and the lens material 125 provides for highly directional emitted light towards the skin with little interference. In addition, the reflected light from the patient is directed in a highly efficient manner through the lens material 125 of the detector cavity to the detector 135. As a result, the device 100 provides for very high gains and minimizes the effects of motion artifacts and interferences with ambient light.

The devices described herein also need not be mechanically coupled to the body to obtain an accurate reading. Because the device need not be in direct contact with the skin and there is no need for mechanical coupling to a patient, the problems that can result including pressure point injuries, pressure necrosis, exsanguinations, discomfort, compression marks, erroneous measurements, infections and other issues caused by direct contact with a device can be avoided.

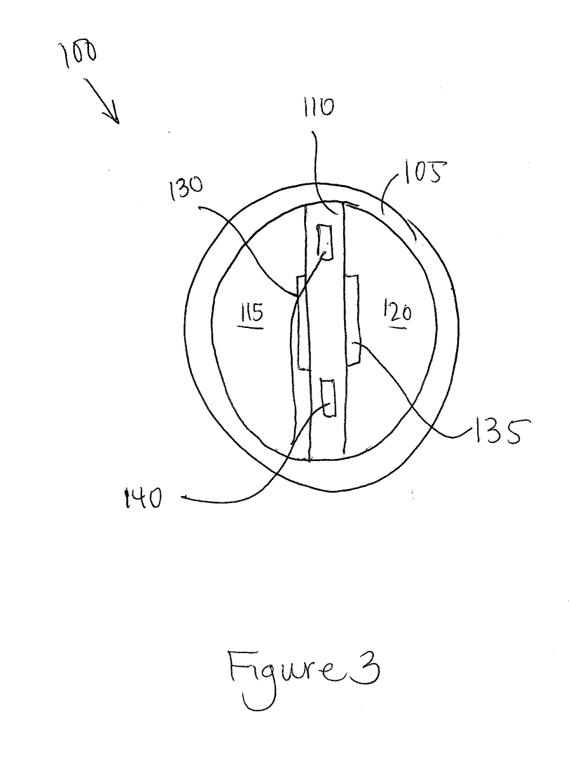

The monitoring device 100 also can include one or more sensors to monitor patient conditions in addition to arterial hemoglobin oxygen saturation, including heart rate or other electrical activity of the heart, volume of individual blood pulsations, or a combination thereof. As best shown in FIG. 3, an additional sensor such as an electrocardiogram (ECG/EKG) electrode 140 can be incorporated in the device 100, such as a Lead II rhythm ECG electrode. The electrode 140 can be positioned on a distal-facing surface of the partition 110. The ECG electrode 140 can also include a piezo electric sensor or a silicone rubber with platinum.

A proximal portion 147 of the housing 105 or the housing 105 itself can be manually gripped by a user or gripped using a tool such as hemostatic clamps. The hemostatic clamp can include handles that can be held in place by a locking mechanism such as a series of interlocking teeth, a few on each handle, that allow a user to adjust the clamping tension of the pliers on the proximal portion 147 or the housing 105. A projection of lens material 125 at the distal end of the housing 105 can then be pressed against a patient to obtain a reading. In further implementations, the housing 105 can be integrated with a hemostatic clamp device. The housing 105 can be gripped by a user (manually or by a tool) and temporarily applied against a patient's body to take periodic readings. It should be appreciated that mechanisms such as suction, although not necessary to obtain accurate readings, can be used.

Again with respect to FIG. 1, the device 100 can communicate with a receiving device 150 via a cable 155, although the connection to the receiving device 150 may be wireless. The receiving device 150 can receive the various signals from the device 100 such as arterial hemoglobin oxygen saturation, heart rate, electrical activity of the heart such as a lead II rhythm ECG, and other measurements to monitor other conditions of the patient as described above. The receiving device 150 can be a computer with a display. The receiving device 150 can also be linked to a handheld ambulatory device either in a wired or wireless manner. The receiving device 150 can include a variety of interfaces or ports that can be used to upload information or download information from the receiving device 150. The receiving device 150 can include softkeys or hard keys so as to interact with the display and define functions of the device at any given time. Alternatively, the receiving device 150 can include a touch screen or other graphical user interface. The receiving device 150 can include a variety of indicators that can be audible or visual to provide a user with information regarding the specific functions, status of the device or other information as is known in the art. The receiving device 150 can be a tabletop device, ambulatory or include a mechanical attachment such that it can connect to an IV stand, hospital bed or other structure.

It should be appreciated that the device can be used in a variety of monitoring situations. As mentioned above, the devices described herein can be pressed against a region of the patient's body through which blood courses. In some implementations, the device 100 can be used internally during surgical procedures or procedures involving trauma to skin, organs, or tissues. In another aspect, the devices described herein can be used for monitoring a fetus during the peripartum process. In some implementations of a method of use during the peripartum process, the fetal scalp (or another presenting part if the fetus is positioned other than head first) is available upon dilation of the mother's cervix (e.g. to approximately 2 cm). In an implementation of use, a physician at the onset of labor can make a vaginal examination to determine the position of the fetus after having broken the amniotic membrane at the opening of the vaginal canal. The physician by tactile contact can determine the most accessible part of the fetus such as the scalp or one ear in case of brow presentation, the nose in case of face presentation, or an arm or a leg in case of a breach or shoulder presentation. The physician then can insert the monitoring device 100 through the vaginal canal and press temporarily a distal end of the device 100 against the presenting part of the fetus. The device 100 can be used periodically during the peripartum process allowing the physician to monitor various conditions, such as the oxygenation of the blood, of the fetus during the entire delivery procedure. In some implementations, the device 100 can be pressed against a patient for at least 2 seconds, 3 seconds, 4 seconds, 5 seconds, 6 seconds or more seconds to obtain a reading.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

What is claimed is:

1. An apparatus comprising:
a housing having an internal volume divided into a first and second cavity by a central opaque partition;
a light source positioned within the first cavity of the housing and coupled to a first side of the partition, wherein the light source comprises a first light emitting diode configured to emit a first wavelength of light and a second light emitting diode configured to emit a second wavelength that is different from the first wavelength of light;
a detector positioned within the second cavity of the housing and coupled to a second side of the partition, wherein the detector is configured to measure at least one of an absorbance, a scattering or a frequency of the first and second wavelengths of light; and
a lens material filling the internal volume of the housing and enclosing the first and second cavities.

2. The apparatus of claim 1, wherein the first wavelength of light is between about 660 nanometers to about 735 nanometers and emitted at a first frequency.

3. The apparatus of claim 2, wherein the second wavelength of light is between about 910 nanometers to about 990 nanometers and emitted at a second frequency that is different from the first frequency.

4. The apparatus of claim 1, wherein the lens material comprises an elastomeric lens material.

5. The apparatus of claim 1, wherein the lens material comprises a dielectric lens material.
